(19) United States
(12) Patent Application Publication
(10) Pub. No.: US 2025/0235498 A1
(43) Pub. Date: Jul. 24, 2025

(54) METHODS OF TREATING OR PREVENTING RESPIRATORY DISEASE WITH HOUTTUYNIA CORDATA THUNB-BASED AEROSOL INHALATION THERAPY

(71) Applicant: National Sun Yat-sen University, Kaohsiung City (TW)

(72) Inventor: Chia-Chen Wang, Kaohsiung City (TW)

(73) Assignee: National Sun Yat-sen University, Kaohsiung City (TW)

(21) Appl. No.: 18/416,903

(22) Filed: Jan. 19, 2024

(51) Int. Cl. A61K 36/78; A61K 9/00; A61P 11/06; A61P 35/00

(52) U.S. Cl. CPC A61K 36/78; A61K 9/008; A61P 11/06; A61P 35/00

(57) ABSTRACT

The present invention relates to a method of treating or preventing respiratory disease, comprising the administration of an effective amount of the composition to a subject with respiratory disease via inhalation, wherein the composition comprises Houttuynia Cordata Thunb or the extracts of Houttuynia Cordata Thunb in the form of an aerosol and/or a spray.

### FIELD OF THE INVENTION

The present invention relates to a method of treating or preventing respiratory disease, comprising the administration of an effective amount of the composition to a subject with respiratory disease, wherein the composition comprises Houttuynia Cordata Thunb or the extracts of Houttuynia Cordata Thunb.

### BACKGROUND OF THE INVENTION

Air pollution has become a serious environmental health risk factor worldwide, posing threats to public health. According to the statistical data released by the World Health Organization (WHO) in 2022, more than 90% of world population breathe polluted air, and more than 7 million premature deaths can be attributed to exposure of air pollution. Exposure to air pollution is both the cause and aggravating factor of many respiratory diseases, including chronic obstructive pulmonary disease (COPD), asthma, and lung cancers.

Chronic respiratory diseases severely affect both children and adults. The presence of unhealthy, potentially harmful and pathogenic fine particulate matters suspended in the air, such as toxic anthropogenic aerosols produced in the air pollution, pathogens, microbes and irritating allergens, may trigger immune responses and development of respiratory and other systemic diseases for people who simply inhale such particulate matters via breathing.

Among various man-made particulate matters in the air produced from anthropogenic activities, phenol aqueous aerosols are relevant as they may be released from phenol usage industry, formed via aerosolization of phenol-contaminated wastewater, or formed directly in the atmosphere when benzene reacts with OH radicals. Phenol is also the precursor to form larger phenolic species, an important family of secondary organic aerosols (SOAs). Phenol can cause mucous and skin irritation on humans and animal upon exposure. Long-term inhalation of industrial pollutants have been corroborated to impair respiratory health and cause airway inflammation.

Ozone is highly oxidizing gaseous pollutant, representing another main source of air pollution. Ozone is widely used in many industries, such as purification air and drinking water, industrial waste treatment, oils, bleaching and waxes. Exposure to ozone may cause headaches, coughing, dry throat, shortness of breath, a heavy feeling in chest, and pulmonary edema in the lungs. Long-term ozone exposure may lead to asthma and COPD.

One of the common respiratory diseases is asthma which results from the maladaptive immune responses to environmental pollutants. The main features of asthma include variable and recurring symptoms, reversible airflow obstruction, and bronchospasm. Common symptoms in asthmatic patients are wheezing, coughing, chest tightness, chest tightness and dyspnea. Though the efficacy of current therapy is effective, such as steroid-related treatment or anti-histamine, the prevalence still increases and those therapies do not cure.

Another common chronic respiratory disorder is COPD, which causes irreversible airways obstruction. According to the statistics from World Health Organization (WHO), COPD is one of the top ten causes of globally death in recent twenty years. COPD is also triggered by long-term exposure environmental pollutants such as particle matters (PM) and irritating gases. The current therapy is bronchodilators. However, the efficacy is limited and the side effects can be severe.

Accordingly, there exists an urgent demand for novel treatments for COPD since the current regimen is not adequate.

### SUMMARY OF THE INVENTION

In view of the above technical circumstances, the present invention discloses a method of treating respiratory disease, comprising the administration of an effective amount of the composition to a subject with certain respiratory distress or disease, wherein the composition comprises Houttuynia Cordata Thunb or the extracts of Houttuynia Cordata Thunb.

In one embodiment, the respiratory disease of the present invention is selected from chronic obstructive pulmonary disease (COPD), asthma, lung fibrosis, cystic fibrosis and acute respiratory distress syndrome.

In one preferred embodiment, the respiratory disease of the present invention is COPD or asthma.

The present invention also discloses a method of treating lung cancer, comprising the administration of an effective amount of the composition to a subject with lung cancer, wherein the composition comprises Houttuynia Cordata Thunb or the extracts of Houttuynia Cordata Thunb.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the schematic drawing of atomized TSI model 9306.

FIG. 2 illustrates the treatment administration experimental procedure of ovalbumin (OVA)-sensitized mice model. (PAA=phenol aqueous aerosols, HCA-A=Houttuynia Cordata Thunb aqueous extract drug A, HCA-B=Houttuynia Cordata Thunb aqueous extract drug B, HCA-C=Houttuynia Cordata Thunb aqueous extract drug C, and NAC=N-acetylcysteine)

FIG. 3 illustrates the detection of cytokine and chemokine in bronchoalveolar lavage fluid (BALF) of OVA-sensitized phenol-induced chronic obstructive pulmonary disease (COPD) animal model.

FIG. 4 illustrates the detection of cytokine and chemokine in BALF of ozone-induced COPD animal model.

FIG. 5 illustrates the detection of cytokine and chemokine in serum of A549 lung cancer animal model.

FIG. 6 illustrates the bioluminescence imaging (BLI) of the progression and metastasis of A549 cells for nude mice injected A549 cells until day 35 post-injection.

FIG. 7 illustrates the BLI values for nude mice injected A549 cells until day 35 post-injection.

### DETAILED DESCRIPTION OF THE INVENTION

Hereinafter, preferred embodiments according to the present disclosure will be described in detail with reference to the accompanying drawings. The detailed description to be disclosed below with the accompanying drawings is intended to describe exemplary embodiments of the present disclosure, and is not intended to represent the only embodiments in which the present disclosure may be practiced. The detailed description below includes specific details to provide a though understanding of the present disclosure. However, those skilled in the art appreciates that the present disclosure may be practiced without these specific details.

As used herein, “a,” “an,” “the,” “at least one,” and “one or more” are used interchangeably.

The present invention discloses a method of treating respiratory diseases, comprising the administration of an effective amount of a composition to a subject with respiratory disease, wherein the composition comprises Houttuynia Cordata Thunb or the extracts of Houttuynia Cordata Thunb.

In one embodiment of the present invention, the composition is formulated and generated in the form of an aerosol or/and a spray.

In one embodiment of the present invention, the composition is first prepared in the form of aqueous solution, and then aerosolized/nebulized by a high flow aerosol generator with backing pressure ranging from 10 to 35 psig.

In one embodiment of the present invention, the size of generated aerosol with designated composition, described in terms of aerodynamic diameter, is ranged from 10 nm to 100 μm.

In one embodiment of the present invention, the respiratory disease of the present invention includes chronic obstructive pulmonary disease (COPD), asthma, lung fibrosis, cystic fibrosis and acute respiratory distress syndrome.

In one embodiment of the present invention, the respiratory disease of the present invention is chosen to be COPD and asthma.

In one preferred embodiment of the present invention, the respiratory disease of the present invention is either COPD or asthma.

In one embodiment of the present invention, the composition treats the respiratory disease by reducing the expression of IL-la.

In one embodiment of the present invention, the composition treats the respiratory disease by reducing the expression of IL-9.

In another embodiment of the present invention, the composition treats the respiratory disease by reducing the expression of vascular endothelial growth factor (VEGF).

In another embodiment of the present invention, the composition treats the respiratory disease by reducing the expression of IFN-γ-inducible protein 10 (IP-10).

The present invention also discloses a method of treating lung cancer, comprising the administration of an effective amount of the composition to a subject with lung cancer, wherein the composition comprises Houttuynia Cordata Thunb or the extracts of Houttuynia Cordata Thunb.

In one embodiment of the present invention, the composition further comprises the glutathione (GSH) and/or butylidenephthalide (BP).

In one embodiment of the present invention, the composition is formulated in the form of an aerosol or/and a spray.

In one embodiment of the present invention, the composition is aerosolized by a high flow aerosol generator with backing pressure ranging from 10 to 35 psig.

In one embodiment of the present invention, the size of aerosol containing the composition is ranged from 10 nm to 100 μm.

In one embodiment of the present invention, the composition treats lung cancer by reducing the expression of Eotaxin.

In one embodiment of the present invention, the composition treats lung cancer by reducing the expression of IL-5.

In one embodiment of the present invention, the composition treats lung cancer by reducing the expression of IL-13.

In one embodiment of the present invention, the composition treats lung cancer by reducing the expression of granulocyte colony-stimulating factor (G-CSF).

In one embodiment of the present invention, the composition treats lung cancer by reducing the expression of IP-10.

### EXAMPLE

The present invention will now be described more specifically with reference to the following examples. It is to be noted that the following descriptions of this invention are presented herein for the purposes of illustration and description only; it is not intended to be exhaustive or to be limited to the precise form disclosed.

### Example 1: Preparation of Aerosol Formulation

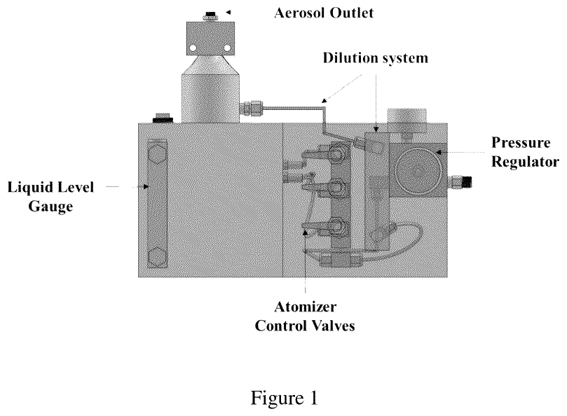

Aerosols of test substances were generated via a steady output liquid feed atomizer (TSI model 9306) for inhalation exposure experiments. As shown in FIG. 1, the device is comprised of four major components: pressure regulator, atomization region, dilution system and aerosol outlet. Synthetic air (79% N₂+21% O₂) was used as the carrier gas with a backing pressure ranging between 10 and 35 psig.

The aerosol jet was formed by expansion of compressed synthetic air with pressure of 10 psi through an orifice with 0.015-inch diameter, which draws liquid up to the atomization area through a narrow tube by a pressure drop. The aerosols within the desired size range can bypass through the impactor and is expelled through a specific outlet, while aerosols or droplets larger than the specified size range hit the spherical impactor and return to the liquid reservoir.

### Example 2: Preparation of Houttuynia Cordata Thunb (H. Cordata) Extract and Houttuynia Cordata Thunb Based Aerosols for Inhalation Therapy

The fresh leaves of Houttuynia Cordata Thunb (270 g) were extracted with deionized water (600 mL) at 70° C. for 1.5 hour. The plant extract was then centrifuged at 4500 rpm for 10 minutes, and further filtered through Advantec Grade No. 1 filter paper (Nominal retention: 6 μm) to remove any residual fine particulates. The plant extract was stored at 4° C. in the dark.

Samples of aerosol drugs were prepared by atomizing Houttuynia Cordata Thunb aqueous (HCA) extract into the aerosol form. Alternatively, aerosol drugs with modified formulation were prepared by dissolving 500 μM Glutathione (GSH), or dissolving 500 μM GSH and 500 μM z-butylidenephthalide (BP) to Houttuynia Cordata Thunb extract solution, respectively in the following phenol-induced chronic obstructive pulmonary disease (COPD) and A549 lung cancer animal models. 0.25M aqueous phenol solution was used to generate phenol aerosols, representing the airborne environmental pollutants. In addition, we prepared N-Acetylcysteine (NAC) aerosol drug by atomizing 500 μM NAC solution into the aerosol form.

### Example 3: Ovalbumin (OVA)-Sensitized Phenol-Induced COPD Animal Model

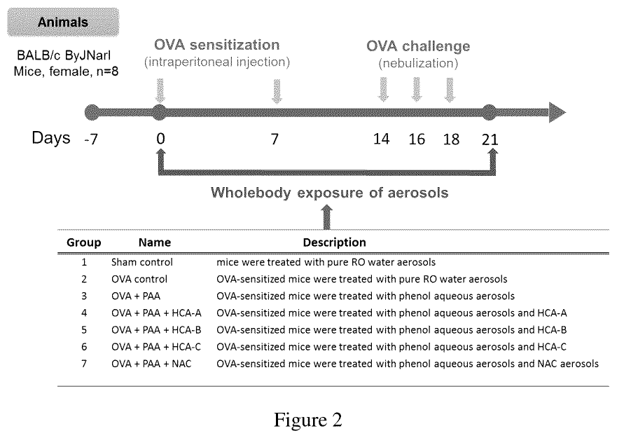

The treatment administration process of OVA-sensitized mice model is shown in FIG. 2. Four types of aerosol drugs with varying formulation are separately named as drug A, drug B, drug C and NAC. Drug A, drug B and drug C are mainly composed by Houttuynia Cordata Thunb aqueous extract. Drug A (HCA-A) is pure Houttuynia Cordata Thunb aqueous extract. Drug B (HCA-B) is comprised of Houttuynia Cordata Thunb aqueous extract and 500 μM GSH, while Drug C (HCA-C) is comprised of Houttuynia Cordata Thunb aqueous extract, 500 M GSH and 500 μM BP. NAC is a mucolytic and antioxidant drug already prevalently used in clinic that are known to modulate several pathways of inflammation. NAC is used here as the positive control to compare the efficacy of aerosol formulation of Houttuynia Cordata Thunb in this study.

The OVA-sensitized mice were firstly placed in a custom-built 40 cm×40 cm×30 cm whole-body acrylic exposure chamber, which was filled and continuously replenished with phenol aqueous aerosols (PAA) for 30 minutes, twice per day for three consecutive weeks. PAA were prepared by atomizing 0.25M aqueous phenol solution via a high flow, liquid-feed atomizer (Model 9306, TSI). PAA such generated exhibit a narrow size distribution with an average size of 22 nm, as measured by a scanning mobility particle sizing system.

Female BALB/c ByJNarl mice (age: 6-8 weeks, weight: 20-25 g) were purchased from National Laboratory Animal Center (NLAC, NARLabs, Taiwan) and approved by the Institutional Animal Care and Use Committee (IACUC) for the experiment of phenol aerosol exposure and inhalation therapy on OVA-treated mice. The mice were randomly divided into seven groups (n=8 per group), including: (1) Sham control: mice were treated with pure RO water aerosols, (2) OVA control: OVA-sensitized mice were treated with pure RO water aerosols, (3) OVA+PAA: OVA-sensitized mice were treated with PAA, (4) OVA+PAA+Drug A group: OVA-sensitized mice were treated with PAA and Houttuynia Cordata Thunb-based aerosols Drug A, (5) OVA+PAA+drug B group: OVA-sensitized mice were treated with PAA and Houttuynia Cordata Thunb-based aerosols Drug B, (6) OVA+PAA+Drug C group: OVA-sensitized mice were treated with PAA and Houttuynia Cordata Thunb-based aerosols drug C, (7) OVA+phenol+NAC group: OVA-treated mice was treated with phenol aqueous aerosols and NAC aerosols. Drug A, Drug B and Drug C designate three Houttuynia Cordata Thunb-based aerosol medicine candidates with minor variation in the formulation as described above. Houttuynia Cordata Thunb-based aerosol medicine exhibits an average size of 100 nm. NAC is an existing drug commonly used to treat numerous lung disorders, and is used here as positive control.

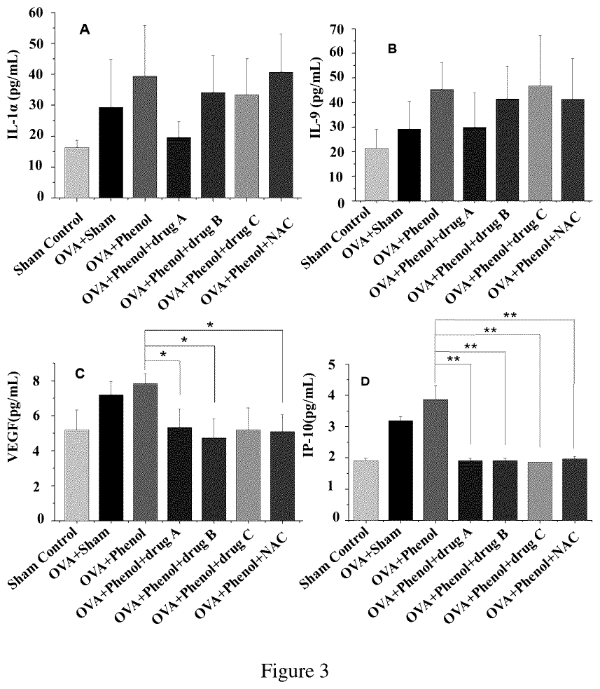

The mice were sacrificed after the 3-week phenol aerosol exposure experiment, and the bronchoalveolar lavage fluid (BALF) of mice was collected for cytokine and chemokine detection, as shown in FIG. 3. For the BALF collection, the left lung was ligated and the right lungs underwent lavage slowly 2 times with a total volume of 0.6 mL ice-cold phosphate buffered saline (PBS). The cells in lavage fluid were removed by centrifugation at 400 g for 5 minutes, and the supernatant was frozen at −80° C. for biomarker analysis. Data are shown as mean±SD (n=4 per group); (P-value: *: p<0.05; **: p<0.01).

As shown in FIG. 3, Interleukin 1α (IL-1α), a highly pleiotropic and potent pro-inflammatory cytokine that activate the inflammatory process, is significantly reduced with aerosol medicine Drug A group. Drug B and Drug C are also effective in reducing the level of IL-1α. In contrast, treatment of the existing drug, NAC in the aerosol form did not show apparent reduction of IL-la.

As shown in FIG. 3, the expression of interleukin-9 (IL-9), a pleiotropic cytokine that has been linked to airway inflammation, mast cell hyperplasia, increased bronchial hyper-responsiveness, allergic asthma, COPD, was significantly suppressed with aerosol medicine Drug A group.

As shown in FIG. 3, the expression of vascular endothelial growth factor (VEGF), an important mediator for angiogenesis, endothelial cell proliferation, migration, and vascular permeability, was significantly reduced in the all three formulations of Houttuynia Cordata Thunb extract-based aerosol medicine group, including Drug A, Drug B and Drug C. VEGF has been suggested to be linked to lung edema, acute and chronic lung diseases, including acute lung injury (ALI), asthma, COPD, pulmonary hypertension, and emphysema.

As shown in FIG. 3, the expression of IFN-γ-inducible protein 10 (IP-10, CXCL10) that is linked to numerous inflammatory diseases, including infectious diseases, immune dysfunction and tumor growth, was significantly down-regulated upon administration of all three formulations of Houttuynia Cordata Thunb extract-based aerosol medicine, including Drug A, Drug B and Drug C (p<0.01).

### Example 4: Ozone-Induced COPD Animal Model

The ozone-induced COPD animal model was performed by an inhalation exposure system. The exposure system mainly composed of ozone generator, equipment of real-time detection of ozone and two 40 cm×40 cm×30 cm chambers made by acrylic. One of the chambers was a mixing chamber which contributes to homogeneous mixing of the gaseous ozone generated from the ozone generator and air, the other one is the whole-body exposure chamber for mice.

Female C57BL/6JNarl mice were purchased from National Laboratory Animal Center (NLAC, NARLabs, Taiwan) for the animal experiment of COPD induced by ozone and inhalation therapy of Houttuynia Cordata Thunb extract-based aqueous aerosol drug. Animals were randomly assigned to the following four groups and five mice for each group. There are four groups: air group (air exposure on normal mice), ozone group (ozone exposure on mice), ozone+Drug B group (inhalation of Drug B on ozone-exposured mice) and Drug B group (inhalation of Drug B on normal mice).

Mice were exposed to 2.5 ppm ozone in the whole-body exposure chamber for eight weeks, three time per week, three hours each time. The aerosol concentration in the whole-body exposure chambers were detected to be 3.54718×10⁵ particles/L. The aerosol dosage of mice through inhalation have been calculated via the following formula:

Dosage=aerosol Concentration*breathing frequency*tidal volume

Breathing frequency and tidal volume in rest have been reported to be 332 breaths/min and 0.2×10⁻³ L. Accordingly, the aerosol dosage of mice through inhalation is calculated to be 23553.2 particles/min.

Experimental mice were sacrificed at the end of the experiment. BALF and blood samples were collected from the mice for pathological analysis.

### Example 5: Setup of Real-Time Detection of Ozone

In order to measure and maintain the ozone concentration during exposure period, a real-time ozone concentration detection system was set-up.

Due to the high absorption of ozone at 254 nm, UV-visible spectroscopy could be used to evaluate the ozone concertation during ozone exposure period. The system was mainly comprised of a UV-visible light source, a glass tube with 90 cm in length, and a detector. A broadband light source (DH-2000 family, Ocean Optics Inc., USA) and a TEC-cooled spectrometer (AvaSpec-2048XL, Avantes, Inc., Holland) were equipped with a back-thinned CCD image sensor (S11155-2048-02, Hamamatsu) and were used to acquire the UV-visible absorption spectra. The ozone concentration was obtained from setup of real-time detection of ozone and calculation by Beer-Lambert law:

−ln(I/I₀)=σ*N*b

In the above equation, o is absorption cross section (cm² molecule⁻¹), b is the absorption path length, N is the number concentration of the absorber (molecule cm⁻³), I is the intensity of transmitted light, I₀ is the intensity of incident light, A is absorbance and T is transmittance. The transmittance could be immediately obtained and the value of absorption cross section and absorption path length were already known. Then, the ozone concentration in the whole-body exposure chamber could be monitored in the ozone-induced COPD experiment.

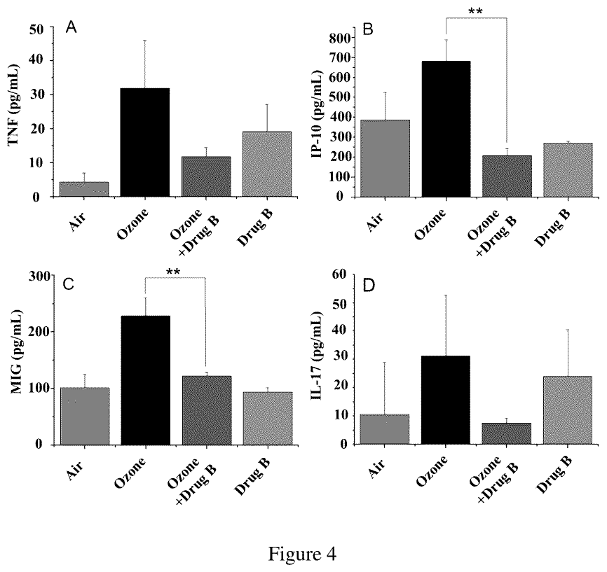

As shown in FIG. 4, TNF-α, a prominent pro-inflammatory cytokine was significantly down-regulated in Houttuynia Cordata Thunb extract-based aerosol medicine, Drug B group.

As shown in FIG. 4, IP-10 was significantly down-regulated in Houttuynia Cordata Thunb extract-based aerosol medicine, Drug B group.

As shown in FIG. 4, MIG was significantly down-regulated in Houttuynia Cordata Thunb extract-based aerosol medicine, Drug B group.

As shown in FIG. 4, IL-17, which induced reactive oxygen species (ROS) production and affected neutrophil infiltration in the respiratory tract and mucus hypersecretion, was down-regulated in Houttuynia Cordata Thunb extract-based aerosol medicine, Drug B group.

The present invention discloses a method of treating respiratory disease, involving the administration of an effective amount of the composition to a subject with respiratory disease, wherein the composition comprises Houttuynia Cordata Thunb or the extracts of Houttuynia Cordata Thunb.

In one embodiment, the respiratory disease of the present invention is selected to be COPD and asthma.

In one preferred embodiment, the respiratory disease of the present invention is COPD or asthma.

### Example 6: Phenol-Induced COPD in A549 Lung Cancer Animal Model

A549/Luc cells were injected into mice to induce lung cancer xenotransplantation. Firstly, the mice were exposed to PAA for three consecutive weeks, twice a day for 30 min each time as mentioned above. After exposure to aerosols, organ tissues, serum samples, and the Bioluminescence imaging (BLI) of tumor of mice were collected for pathological analysis.

Male CAnN.Cg-Foxn1^{nu}/CrlNarl mice (age: 7-9 weeks, weight: 18.7-26.3 g) were purchased from National Laboratory Animal Center, National Applied Research Laboratories (NLAC, NARLabs, Taiwan) and approved by the Institutional Animal Care and Use Committee (IACUC). They were maintained under controlled conditions of RH of 30-70%, a 12-hour light/dark cycle, and at 23±2° C. for one week before the experiment began.

In the lung cancer induced model, mice were inoculated through caudal vein with a single dose of 1×10⁵ A549/Luc 2 cells (ATCC, USA) to develop lung cancer model. After cancer cell injection into the tail veins, the animals were randomly assigned to the following six groups for the tests. Normal Control group (mice solely exposed to RO water aerosol, n=8), A549 group (mice were implanted A549 cells and exposed to RO water aerosol, n=8), A549+Phenol group (mice were implanted A549 cells and exposed to 0.25M phenol aerosols, n=8), A549+Phenol+Drug A (mice were implanted A549 cells and exposed to 0.25M phenol aerosols, followed by treatment of Drug A, n=8), A549+Phenol+Drug B (mice were implanted A549 cells and exposed to 0.25M phenol aerosols, followed by treatment of Drug B, n-8), A549+Phenol+Drug C (mice were implanted A549 cells and exposed to 0.25M phenol aerosols, followed by treatment of Drug C, n=8).

Three different formulations of Houttuynia Cordata Thunb-based aerosol medicine that were prepared and administered to the A549 mice. The formulation of aerosol medicine drug A, aerosol medicine drug B and aerosol medicine drug C were Houttuynia Cordata Thunb leaves extract, Houttuynia Cordata Thunb extract mixed with 500 μM GSH, and Houttuynia Cordata Thunb extract mixed with 500 μM GSH and 500 μM BP, respectively.

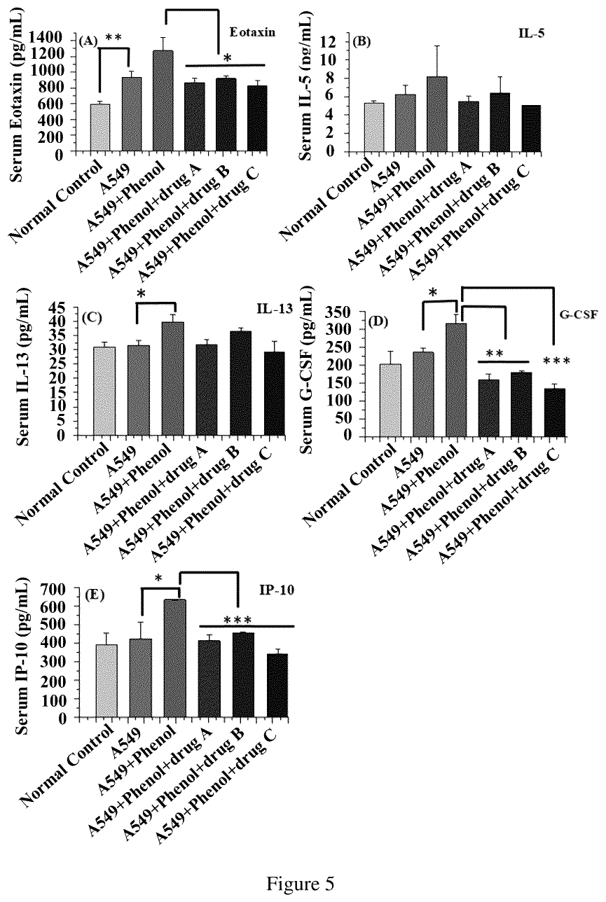

As shown in FIG. 5 (A), exotoxin was significantly down-regulated in all three groups treated with Houttuynia Cordata Thunb extract-based aerosol medicine group, as compared with the A549+Phenol group (P-value: *: p<0.05).

As shown in FIG. 5 (B), IL-5 was reduced in all three groups treated with Houttuynia Cordata Thunb extract-based aerosol medicine group, as compared with the A549+Phenol group.

As shown in FIG. 5 (C), IL-13 was reduced in all three groups treated with Houttuynia Cordata Thunb extract-based aerosol medicine group, as compared with the A549+Phenol group.

As shown in FIG. 5 (D), granulocyte colony-stimulating factor (G-CSF) was significantly down-regulated in all three groups treated with Houttuynia Cordata Thunb extract-based aerosol medicine group, as compared with the A549+Phenol group (P-value for the treatment of Drug A and Drug B: **: p<0.01; P-value for the treatment of Drug C: ***: p<0.005).

As shown in FIG. 5, IP-10 was significantly down-regulated in all three groups treated with Houttuynia Cordata Thunb extract-based aerosol medicine group, as compared with the A549+Phenol group (P-value for the treatment of Drug A, Drug B, Drug C: ***: p<0.005).

### Example 7: Tumor Progression Analysis of Bioluminescence Imaging (BLI)

BLI can monitor the status or progression of a disease in a non-invasive manner and has been applied in probing the tumor growth and metastasis. In the lung cancer model used in this study, luciferase-tagged A549 human lung adenocarcinoma cells were injected, and animal imaging is used to assess the growth and metastasis of lung tumors. Under the firefly luciferase-catalyzed luciferin reaction, the light emission is broadband (530-640 nm).

BLI was analyzed 1-5 weeks post-tumor injection. BLI was measured on the Ami HTX optical imaging system (Model Ami HT, Spectral Instruments Imaging., USA). BLI was performed by a highly sensitive, cooled Charge Coupled Device (CCD) sensor. Imaging and quantification of signals were controlled by the acquisition and analysis software Spectral Instruments Imaging. Luminescence, and fluorescence, and x-ray modalities were combined into the system. The luminescence value obtained through the CCD captured the luminescence photons and the photons were converted into electronic signals through the photomultiplier tube (PMT), and then calculated through the software Spectral Instruments Imaging. According to the LUC2 gene phenotype of A549 cells, the fluorescence signals of A549 cells was observed at a wavelength of 590 nm. For the in vivo imaging, mice were anesthetized and placed onto the heated platform inside the light-tight camera box. Imaging time ranged from 1 s to 5 min, depending on the luminescence signals.

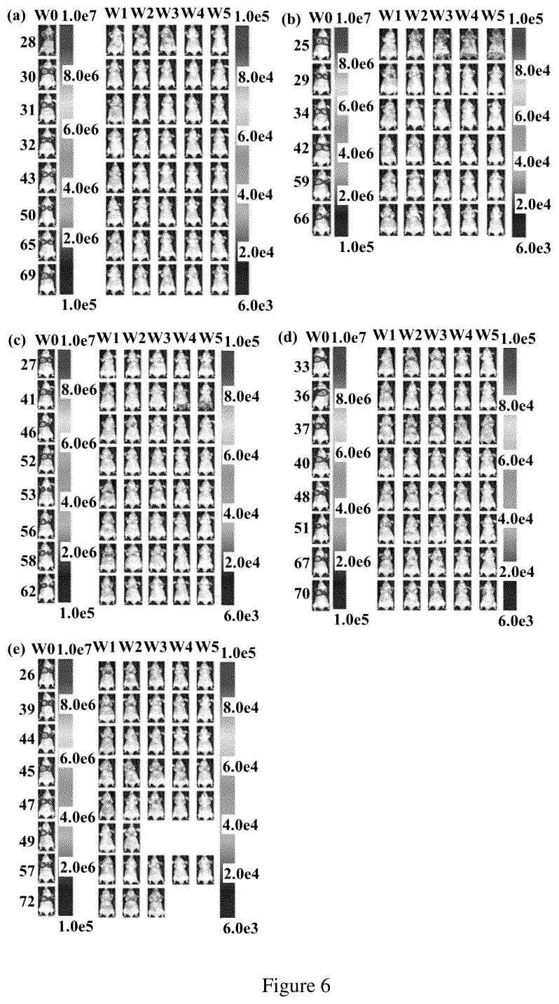

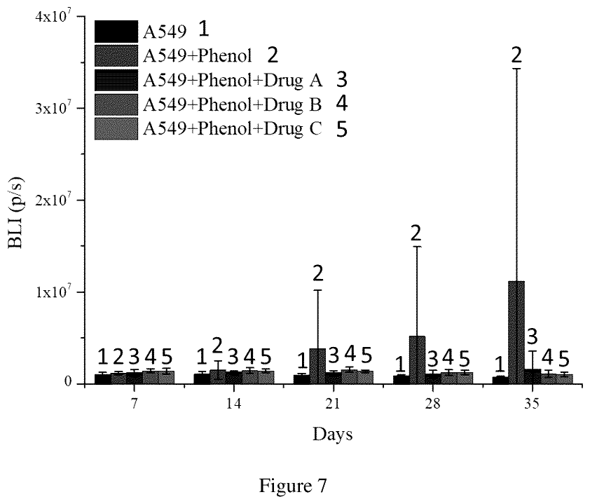

As shown in FIG. 6 and FIG. 7, Houttuynia Cordata Thunb extract-based aerosol medicine with BP and GSH group suppressed the growth of lung cancer cells.

The present invention also discloses a method of treating lung cancer, comprising the administration of an effective amount of the composition in the aerosol form to a subject with lung cancer, wherein the composition comprises Houttuynia Cordata Thunb or the extracts of Houttuynia Cordata Thunb.

While the invention has been described and exemplified in sufficient details for those skilled in this art to make and use it, various alternatives, modifications, and improvements should be apparent without departing from the spirit and scope of this invention.

One skilled in the art readily appreciates that the present invention is well adapted to carry out the objects and obtain the results and efficacy mentioned, as well as those inherent therein. The processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method of treating respiratory disease, comprising the administration of an effective amount of a composition to a subject with respiratory disease, wherein the composition comprises Houttuynia Cordata Thunb or the extracts of Houttuynia Cordata Thunb.

2. The method of claim 1, wherein the composition is formulated and generated in the form of an aerosol or/and a spray.

3. The method of claim 2, wherein the composition is aerosolized by a high flow aerosol generator with backing pressure ranging from 10 to 35 psig.

4. The method of claim 2, wherein the size of aerosol containing the composition is ranged from 10 nm to 100 μm.

5. The method of claim 1, wherein the respiratory disease includes chronic obstructive pulmonary disease, asthma, lung fibrosis, cystic fibrosis and acute respiratory distress syndrome.

6. The method of claim 1, wherein the composition treats the respiratory disease by reducing the expression of IL-la.

7. The method of claim 1, wherein the composition treats the respiratory disease by reducing the expression of IL-9.

8. The method of claim 1, wherein the composition treats the respiratory disease by reducing the expression of vascular endothelial growth factor (VEGF).

9. The method of claim 1, wherein the composition treats the respiratory disease by reducing the expression of IFN-γ-inducible protein 10 (IP-10).

10. A method of treating lung cancer, comprising the administration of an effective amount of the composition to a subject with lung cancer, wherein the composition comprises Houttuynia Cordata Thunb or the extracts of Houttuynia Cordata Thunb.

11. The method of claim 10, wherein the composition further comprises the glutathione (GSH) and/or butylidenephthalide (BP).

12. The method of claim 10, wherein the composition is formulated in the form of an aerosol or/and a spray.

13. The method of claim 11, wherein the composition is aerosolized by a high flow aerosol generator with backing pressure ranging from 10 to 35 psig.

14. The method of claim 11, wherein the size of aerosol containing the composition is ranged from 10 nm to 100 μm.

15. The method of claim 11, wherein the composition treats lung cancer by reducing the expression of Eotaxin.

16. The method of claim 11, wherein the composition treats lung cancer by reducing the expression of IL-5.

17. The method of claim 11, wherein the composition treats lung cancer by reducing the expression of IL-13.

18. The method of claim 11, wherein the composition treats lung cancer by reducing the expression of granulocyte colony-stimulating factor (G-CSF).

19. The method of claim 11, wherein the composition treats lung cancer by reducing the expression of IP-10.
